(19) United States
(12) Patent Application Publication
(10) Pub. No.: US 2019/0313986 A1
(43) Pub. Date: Oct. 17, 2019

(54) SYSTEMS AND METHODS FOR AUTOMATED DETECTION OF OBJECTS WITH MEDICAL IMAGING

(71) Applicant: THE GENERAL HOSPITAL CORPORATION, Boston, MA (US)

(72) Inventor: Synho Do, Lexington, MA (US)

(21) Appl. No.: 16/349,717

(22) PCT Filed: Nov. 16, 2017
(86) PCT No.: PCT/US17/61993

§ 371 Date: May 14, 2019

Related U.S. Application Data
(60) Provisional application No. 62/422,952, filed on Nov. 16, 2016.

(51) Int. Cl. A61B 6/12; G06T 7/73; A61B 34/20; A61B 6/02; A61B 6/00; A61B 6/03

(52) U.S. Cl. CPC A61B 6/12; G06T 7/74; A61B 34/20; A61B 6/025; A61B 5/05; A61B 6/032; A61B 6/037; G06T 2207/30021; A61B 6/5211; A61B 6/4441

(57) ABSTRACT

A system may identify the location of objects of interest in a captured image by processing image data associated with the captured image using neural networks. The image data may be generated by an image sensor, which may be part of an imaging system. A cascade segmentation artificial intelligence that includes multiple neural networks may be used to process the image data in order to determine the locations objects of interest in the captured image. Post-processing may be performed on outputs of the cascade segmentation artificial intelligence to generate a mask corresponding to the locations of the objects of interest. The mask may be superimposed over the captured image to produce an output image, which may then be presented on a display.

### CROSS-REFERENCE TO RELATED APPLICATIONS

This application is based on, claims priority to, and incorporates herein by reference in its entirety U.S. Provisional Application Ser. No. 62/422,952, filed Nov. 16, 2016.

### STATEMENT REGARDING FEDERALLY SPONSORED RESEARCH

N/A

### BACKGROUND

The present disclosure relates generally to imaging and, more particularly, to system and methods identifying objects in captured images.

Imaging is important to a wide range of industries and activities. From space exploration to oil exploration, imaging plays a key role in these endeavors. The modalities available for imaging are at least as diverse as the industries that employ them. For example, in the medical industry alone, a staggeringly large number of imaging modalities are employed in regular, clinical medicine. For example, to name but a few, x-ray radiography, magnetic resonance imaging (MRI), computed tomography (CT) imaging, emission tomography imaging (including modalities such as positron emission tomography and single photon emission computed tomography), optical, x-ray fluoroscopy, and many, many others are utilized each day in modern medicine.

It is within this context that embodiments of the present invention arise.

### SUMMARY OF THE DISCLOSURE

The present disclosure provides systems and methods for identifying specific objects in medical images. As will be described, the systems and methods provide greater flexibility and improved results than traditional object identification systems and methods.

In accordance with one aspect of the disclosure, a medical imaging system may include an image sensor, a processor, and a display. The image sensor may be configured to acquire image data from a patient to produce a captured image. The processor may be configured to receive the image data from the image sensor, to determine a location of a peripherally inserted central catheter (PICC) line in the image, and to generate an output image in which the location of the PICC line is highlighted. The display may be configured to display the output image.

In some embodiments, the image sensor may include at least one of a radio frequency (RF) system of a magnetic resonance imaging (MRI) system, an x-ray detector of a computed tomography (CT) system, and a gamma ray detector of an emission tomography system.

In some embodiments, the processor may be configured to determine the location of the PICC line using a first trained neural network, to determine a region of interest for a location of a tip of the PICC line using a second trained neural network, to determine the location of the tip of the PICC line based on the location of the PICC line and the region of interest, and to generate a mask that includes the location of the tip of the PICC line, the location of the region of interest, and the location of the PICC line, wherein the output image comprises the mask superimposed over the captured image.

In accordance with another aspect of the disclosure, a system may include an input and a processor. The input may be configured to receive image data from an imaging system configured to generate the image data. The image data may correspond to a captured image. The processor may be configured to receive the image data from the input, to determine a location of a PICC line in the captured image, and to generate an output image in which the location of the PICC line is highlighted.

In some embodiments, the processor may be configured to determine the location of the PICC line by processing the image data with a first neural network to produce a PICC line prediction image. The first neural network may include a fully convolutional network that includes multiple convolutional layers.

In some embodiments, the processor may be configured to determine the location of a region of interest (ROI) for a location of a tip of the PICC line by processing the image data with a second neural network to produce a ROI prediction image. The first and second neural networks may be included in a cascade segmentation artificial intelligence. The processor may be configured to apply a Hough transform to the PICC line prediction image to produce a filtered PICC line prediction image. The processor may be configured to determine the location of the tip of the PICC line based on the filtered PICC line prediction image and the ROI prediction image. The processor may be configured to produce an output image by generating a mask based on the filtered PICC line prediction image, the ROI prediction image, and the determined location of the tip of the PICC line, and by superimposing the mask over the captured image to produce the output image.

In some embodiments, the imaging system may include at least one of a RF system of a MRI system, an x-ray detector of a CT system, and a gamma ray detector of an emission tomography system.

In accordance with yet another aspect of the disclosure, a method may include generating, with an image system, image data that corresponds to a captured image, receiving, with a processor, the image data from the image system, and executing, with the processor, instructions for determining a location of a PICC line in the captured image and generating an output image in which the location of the PICC line is highlighted.

In some embodiments, determining the location of the PICC line in the captured image includes determining the location of the PICC line in the captured image by processing the image data with a first neural network to produce a PICC line prediction image. The method may further include executing, with the processor, instructions for determining a location of a ROI for a location of a tip of the PICC line by processing the image data with a second neural network to produce a ROI prediction image, wherein the first and second neural networks are included in a cascade segmentation artificial intelligence. The first neural network and the second neural network may include fully convolutional neural networks that each include multiple convolutional layers.

In some embodiments, the method may further include executing, with the processor, instructions for applying a Hough transform to the PICC line prediction image to produce a filtered PICC line prediction image, for determining the location of the tip of the PICC line based on the filtered PICC line prediction image and the ROI prediction image, for generating a mask based on the filtered PICC line prediction image, the ROI prediction image, and the determined location of the tip of the PICC line, and for superimposing the mask over the captured image to produce the output image.

The foregoing and other aspects and advantages of the invention will appear from the following description. In the description, reference is made to the accompanying drawings which form a part hereof, and in which there is shown by way of illustration a preferred embodiment of the invention. Such an embodiment does not necessarily represent the full scope of the invention, however, and reference is made therefore to the claims and herein for interpreting the scope of the invention.

### BRIEF DESCRIPTION OF THE DRAWINGS

FIGS. 1A and 1B show system diagrams of an illustrative x-ray computed tomography (CT) imaging system in accordance with an embodiment.

FIGS. 2A and 2B show system diagrams of another illustrative x-ray CT imaging system in accordance with an embodiment.

FIG. 3 shows a system diagram of an illustrative magnetic resonance imaging (MRI) system in accordance with an embodiment.

FIG. 4 shows an array of images showing chest x-rays of patients having Peripherally Inserted Central Catheter (PICC) lines, which may be analyzed in accordance with an embodiment.

FIG. 5 shows an illustrative process flow diagram representing a system architecture for object location identification in accordance with an embodiment.

FIG. 6A shows an illustrative process flow diagram representing a system architecture for object location identification in accordance with an embodiment.

FIG. 6B shows an illustrative process flow diagram demonstrating post-processing that may be performed as part of a system architecture for object location identification in accordance with an embodiment.

FIG. 7 shows a chest x-ray image and image patches obtained from the chest x-ray image that include portions of objects of interest in accordance with an embodiment.

FIG. 8 shows an array of images that illustrates results of object location identification systems and techniques, showing original images, ground truth labels derived from the original images, and superimposition of the ground truth labels over the original images in accordance with an embodiment.

### DETAILED DESCRIPTION

The systems and methods of the present invention can be utilized with a wide variety of data and systems and methods for acquiring and processing data. Some non-limiting examples of imaging systems follow hereafter. However, the systems and methods of the present disclosure are not limited to these modalities or imaging.

As will be described, in one aspect, the present disclosure provides systems and methods for automatically identifying the location of objects in medical images. This stands in contrast to traditional manual identification of object locations in medical images, which are often time consuming and subject to human error. The present disclosure, provides systems and methods that are not limited in this manner. A framework is provided that can be leveraged to identify the locations of target objects in medical images and to highlight the locations of these target objects using superimposed masks.

For example, machine intelligence techniques utilizing neural networks may quickly and accurately aid in the interpretation of medical images by automatically identifying the locations of important objects in these images, and clearly indicating these locations to a medical professional through the application of superimposed masks.

A primary example of medical image interpretation that could be aided through the use of machine intelligence techniques involves the determination of the location of peripherally inserted central catheter (PICC) lines in chest x-ray radiographs. A PICC line is a thin, flexible plastic tube that provides medium-term intravenous access. PICC lines are generally inserted into arm veins and threaded through the subclavian vein into the superior vena cava (SVC) with the catheter tip directed inferiorly and ideally at the junction of the SVC and the right atrium (RA). Malpositioned PICC lines can have potentially serious complications such as thrombus formation or cardiac arrhythmia. As a result, PICC positioning is always confirmed with a chest x-ray radiograph (CXR) immediately after insertion. This radiograph requires timely and accurate interpretation by a radiologist. Although the error rate for radiologists misinterpreting PICC line locations is generally low, delays in treatment initiation can be substantial (e.g., up to 176 minutes in some cases), particularly when the radiograph is part of a long queue of radiographs to be interpreted. By using computer-aided detection to automatically identify the locations of PICC lines in these radiographs, the speed with which radiographs may be analyzed by radiologists may be increased and the accuracy of diagnoses made based on these analyses may be improved.

The systems and methods provided herein may be used in any of a variety of setting where one looks to automatically identify the locations of one or more target objects in medical images. The systems and methods of the present disclosure are not limited to applications of PICC line location identification, but may be used to detect a variety of other classes of objects in medical images, such as threads, tubes, electrocardiogram (ECG) lines, medical implants, or disease. This and other points will be made clear with respect to the following description. However, before turning to the specifics of the present systems and methods, some non-limiting examples of operational environments, such as imaging systems are provided.

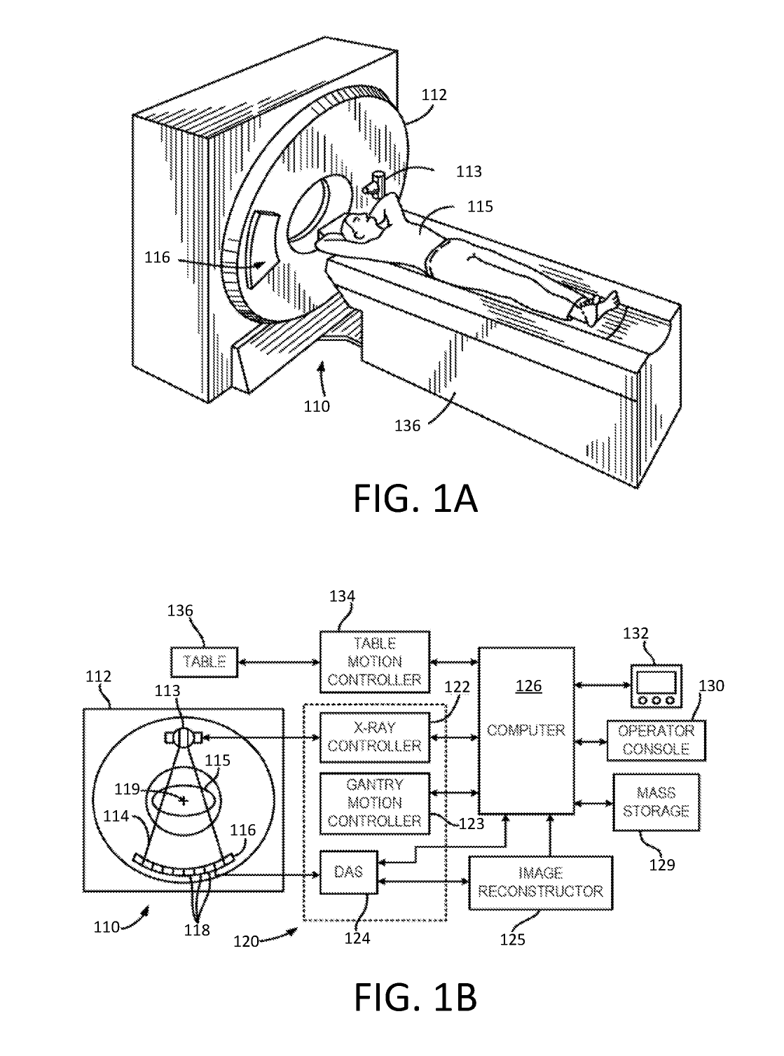

The systems and methods described herein can be used with a variety of medical imaging systems. For example, the systems and methods described herein may be used with traditional x-ray fluoroscopy or with more advanced imaging systems, such as employ computed tomography or tomosynthesis. With initial reference to FIGS. 1A and 1B, an x-ray computed tomography (CT) imaging system 110 includes a gantry 112 representative of a “third generation” CT scanner. Gantry 112 has an x-ray source 113 that projects a fan beam, or cone beam, of x-rays 114 toward a detector array 116 on the opposite side of the gantry. The detector array 116 is formed by a number of detector elements 118 which together sense the projected x-rays that pass through a medical patient 115. Each detector element 118 produces an electrical signal that represents the intensity of an impinging x-ray beam and hence the attenuation of the beam as it passes through the patient. As will be described, this acquired attenuation data of a CT system 110 can be referred to as “sensor data.” In the case of CT imaging, such data is typically in Radon space and measured in hounsfield units. In this way, such sensor data can be referred to as being acquired in a “sensor domain.” In the case of CT imaging and its respective sensor domain, the sensor data must be transformed to an image domain, such as by using filtered backprojection, to yield a reconstructed image. However, as will be described, constraining reconstruction or acquisition based on such traditional tools for domain transfer and their inherent limitations is not necessary. Thus, as will be explained, breaking from this traditional paradigm of CT image reconstruction can yield, in accordance with the present disclosure, superior images.

During a scan to acquire x-ray projection data, the gantry 112 and the components mounted thereon rotate about a center of rotation 119 located within the patient 115. The rotation of the gantry and the operation of the x-ray source 113 are governed by a control mechanism 120 of the CT system. The control mechanism 120 includes an x-ray controller 122 that provides power and timing signals to the x-ray source 113 and a gantry motor controller 123 that controls the rotational speed and position of the gantry 112. A data acquisition system (DAS) 124 in the control mechanism 120 samples analog data from detector elements 118 and converts the data to digital signals for subsequent processing. An image reconstructor 125, receives sampled and digitized x-ray data from the DAS 124 and performs high speed image reconstruction. The reconstructed image is applied as an input to a computer 126 which stores the image in a mass storage device 128.

The computer 126 also receives commands and scanning parameters from an operator via console 130 that has a keyboard. An associated display 132 allows the operator to observe the reconstructed image and other data from the computer 126. The operator supplied commands and parameters are used by the computer 126 to provide control signals and information to the DAS 124, the x-ray controller 122 and the gantry motor controller 123. In addition, computer 126 operates a table motor controller 134 which controls a motorized table 136 to position the patient 115 in the gantry 112.

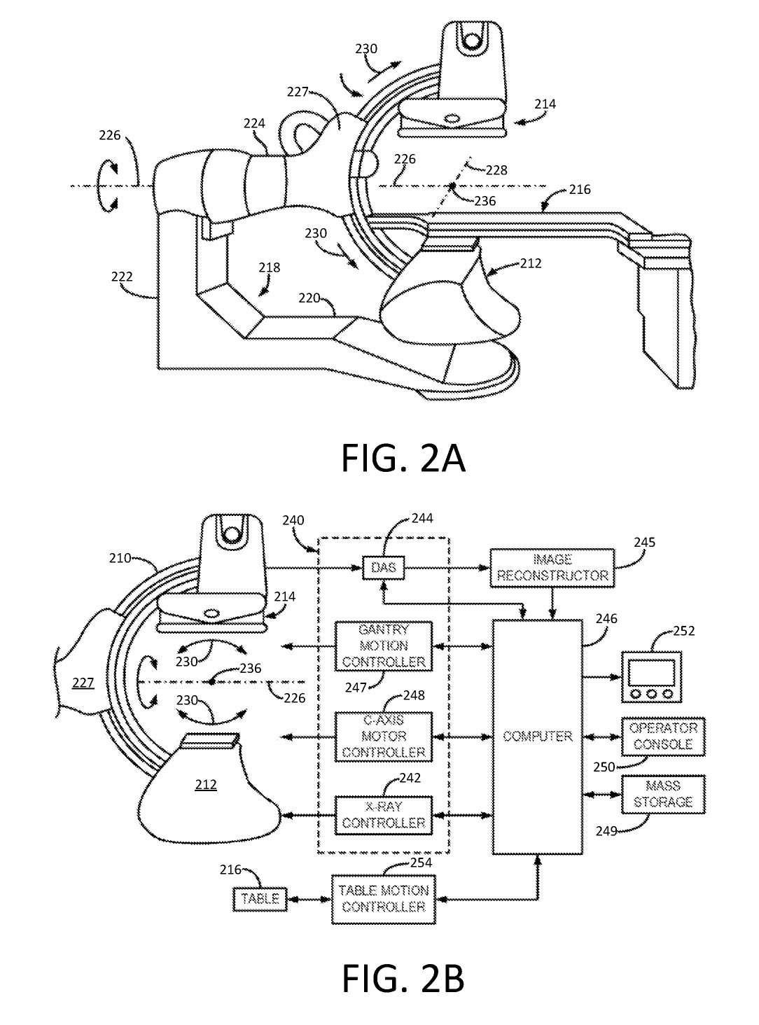

Referring particularly to FIGS. 2A and 2B, the system and method of the present disclosure may be employed to reconstruct images employs an x-ray system that is designed for use in connection with interventional procedures. It is characterized by a gantry having a C-arm 210 which carries an x-ray source assembly 212 on one of its ends and an x-ray detector array assembly 214 at its other end. Similarly to the above-described CT system 110, the data acquired by the C-arm system illustrated in FIGS. 2A and 2B can be referred to as “sensor data,” in this case, typically, acquired in Radon space and measured in hounsfield units.

The gantry enables the x-ray source 212 and detector 214 to be oriented in different positions and angles around a patient disposed on a table 216, while enabling a physician access to the patient. The gantry includes an L-shaped pedestal 218 which has a horizontal leg 220 that extends beneath the table 216 and a vertical leg 222 that extends upward at the end of the horizontal leg 220 that is spaced from of the table 216. A support arm 224 is rotatably fastened to the upper end of vertical leg 222 for rotation about a horizontal pivot axis 226. The pivot axis 226 is aligned with the centerline of the table 216 and the arm 224 extends radially outward from the pivot axis 226 to support a C-arm drive assembly 227 on its outer end. The C-arm 210 is slidably fastened to the drive assembly 227 and is coupled to a drive motor (not shown) which slides the C-arm 210 to revolve it about a C-axis 228 as indicated by arrows 230. The pivot axis 226 and C-axis 228 intersect each other at an isocenter 236 located above the table 216 and they are perpendicular to each other.

The x-ray source assembly 212 is mounted to one end of the C-arm 210 and the detector array assembly 214 is mounted to its other end. As will be discussed in more detail below, the x-ray source 212 emits a cone beam of x-rays which are directed at the detector array 214. Both assemblies 212 and 214 extend radially inward to the pivot axis 226 such that the center ray of this cone beam passes through the system isocenter 236. The center ray of the cone beam can thus be rotated about the system isocenter around either the pivot axis 226 or the C-axis 228, or both during the acquisition of x-ray attenuation data from a subject placed on the table 216.

Referring particularly to FIG. 2B, the rotation of the assemblies 212 and 214 and the operation of the x-ray source 232 are governed by a control mechanism 240 of the CT system. The control mechanism 240 includes an x-ray controller 242 that provides power and timing signals to the x-ray source 232. A data acquisition system (DAS) 244 in the control mechanism 240 samples data from detector elements 238 and passes the data to an image reconstructor 245. The image reconstructor 245, receives digitized x-ray data from the DAS 244 and performs high speed image reconstruction. The reconstructed image is applied as an input to a computer 246 which stores the image in a mass storage device 249 or processes the image further.

The control mechanism 240 also includes pivot motor controller 247 and a C-axis motor controller 248. In response to motion commands from the computer 246 the motor controllers 247 and 248 provide power to motors in the x-ray system that produce the rotations about respective pivot axis 226 and C-axis 228. A program executed by the computer 246 generates motion commands to the motor drives 247 and 248 to move the assemblies 212 and 214 in a prescribed scan path.

The computer 246 also receives commands and scanning parameters from an operator via console 250 that has a keyboard and other manually operable controls. An associated cathode ray tube display 252 allows the operator to observe the reconstructed image and other data from the computer 246. The operator supplied commands are used by the computer 246 under the direction of stored programs to provide control signals and information to the DAS 244, the x-ray controller 242 and the motor controllers 247 and 248. In addition, computer 246 operates a table motor controller 254 which controls the motorized table 216 to position the patient with respect to the system isocenter 236.

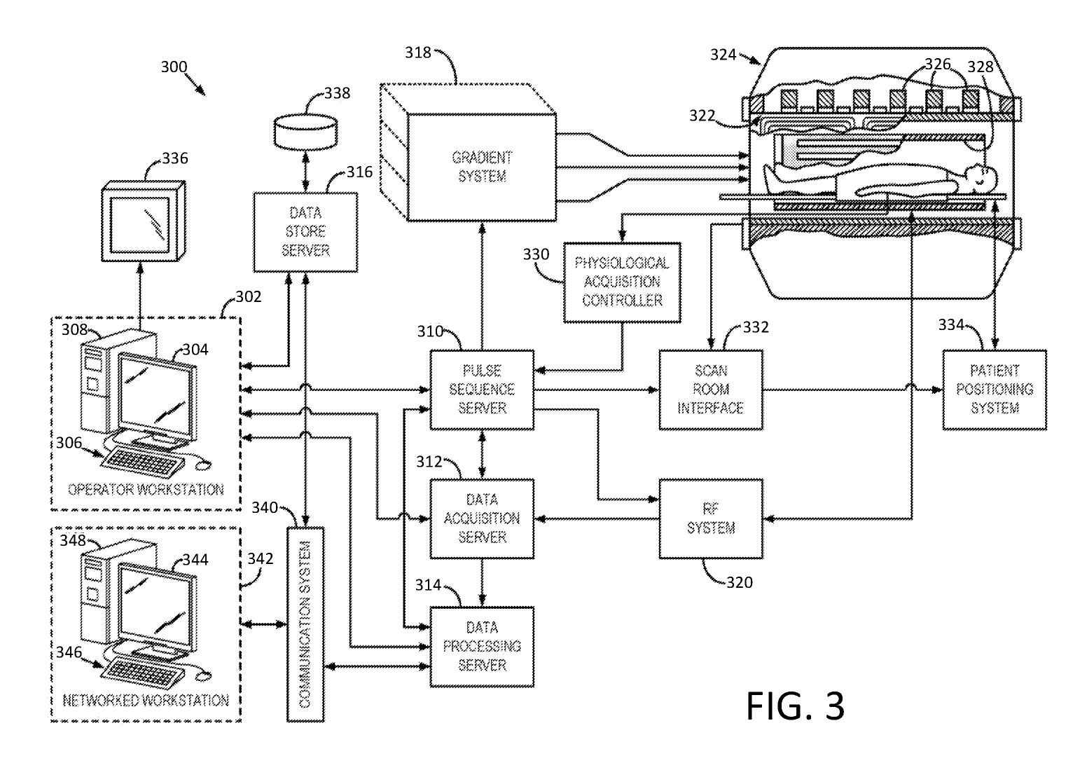

The system and methods of the present disclosure can also be applied to MR imaging systems. Referring to FIG. 3, an example of an MRI system 300 is illustrated. The MRI system 300 includes a workstation 302 having a display 304 and a keyboard 306. The workstation 302 includes a processor 308 that is commercially available to run a commercially-available operating system. The workstation 302 provides the operator interface that enables scan prescriptions to be entered into the MRI system 300. The workstation 302 is coupled to four servers: a pulse sequence server 310; a data acquisition server 312; a data processing server 314; and a data store server 316. The workstation 302 and each server 310, 312, 314, and 316 are connected to communicate with each other.

The pulse sequence server 310 functions in response to instructions downloaded from the workstation 302 to operate a gradient system 318 and a radiofrequency (RF) system 320. Gradient waveforms necessary to perform the prescribed scan are produced and applied to the gradient system 318, which excites gradient coils in an assembly 322 to produce the magnetic field gradients Gₓ, G_{y}, and G_{z} used for position encoding MR signals. The gradient coil assembly 322 forms part of a magnet assembly 324 that includes a polarizing magnet 326 and a whole-body RF coil 328 and/or local coil.

RF excitation waveforms are applied to the RF coil 328, or a separate local coil, such as a head coil, by the RF system 320 to perform the prescribed magnetic resonance pulse sequence. Responsive MR signals detected by the RF coil 328, or a separate local coil, are received by the RF system 320, amplified, demodulated, filtered, and digitized under direction of commands produced by the pulse sequence server 310. The RF system 320 includes an RF transmitter for producing a wide variety of RF pulses used in MR pulse sequences. The RF transmitter is responsive to the scan prescription and direction from the pulse sequence server 310 to produce RF pulses of the desired frequency, phase, and pulse amplitude waveform. The generated RF pulses may be applied to the whole body RF coil 328 or to one or more local coils or coil arrays.

The RF system 320 also includes one or more RF receiver channels. Each RF receiver channel includes an RF preamplifier that amplifies the MR signal received by the coil 328 to which it is connected, and a detector that detects and digitizes the quadrature components of the received MR signal. The magnitude of the received MR signal may thus be determined at any sampled point by the square root of the sum of the squares of the I and Q components:

M=\sqrt{I²+Q²} (1),

and the phase of the received MR signal may also be determined:

$$ϕ = {tan}^{- 1} ( \frac{Q}{I} ) . \qquad ( 2 )$$

In the case of an MRI system 300, these acquired RF signals are sampled in “k-space,” which is a frequency domain. Thus, the MRI system 300 acquires “sensor data” in the frequency domain, which represents the “sensor domain” for MR or NMR imaging. Such MR sensor data must be transformed to an image domain to yield a reconstructed image, which is traditionally achieved via a Fourier transform or projection reconstruction technique.

The pulse sequence server 310 also optionally receives patient data from a physiological acquisition controller 330. The controller 330 receives signals from a number of different sensors connected to the subject to be scanned, such as electrocardiograph (ECG) signals from electrodes, or respiratory signals from a bellows or other respiratory monitoring device. Such signals are typically used by the pulse sequence server 310 to synchronize, or “gate,” the performance of the scan with the subject's heart beat or respiration.

The pulse sequence server 310 also connects to a scan room interface circuit 332 that receives signals from various sensors associated with the condition of the patient and the magnet system. A patient positioning system 332 may be included.

The digitized MR signal samples produced by the RF system 320 are received by the data acquisition server 312. The data acquisition server 312 operates in response to instructions downloaded from the workstation 302 to receive the real-time MR data and provide buffer storage, such that no data is lost by data overrun. In some scans, the data acquisition server 312 does little more than pass the acquired MR data to the data processor server 314. However, in scans that require information derived from acquired MR data to control the further performance of the scan, the data acquisition server 312 is programmed to produce such information and convey it to the pulse sequence server 310. For example, during prescans, MR data is acquired and used to calibrate the pulse sequence performed by the pulse sequence server 310. Also, navigator signals may be acquired during a scan and used to adjust the operating parameters of the RF system 320 or the gradient system 318, or to control the view order in which k-space data (e.g., frequency domain data) is sampled. In all these examples, the data acquisition server 312 acquires MR data and processes it in real-time to produce information that is used to control the scan.

The data processing server 314 receives MR data from the data acquisition server 312 and processes it in accordance with instructions downloaded from the workstation 302. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 314 are conveyed back to the workstation 302 where they are stored. Real-time images are stored in a data base memory cache (not shown), from which they may be output to operator display 304 or a display 336 that is located near the magnet assembly 324 for use by attending physicians. Batch mode images or selected real time images are stored in a host database on disc storage 338. When such images have been reconstructed and transferred to storage, the data processing server 314 notifies the data store server 316 on the workstation 302. The workstation 302 may be used by an operator to archive the images, produce films, or send the images via a network or communication system 340 to other facilities that may include other networked workstations 342.

The communication system 340 and networked workstation 342 may represent any of the variety of local and remote computer systems that may be included within a given imaging facility including the system 300 or other, remote location that can communicate with the system 300. In this regard, the networked workstation 342 may be functionally and capably similar or equivalent to the operator workstation 302, despite being located remotely and communicating over the communication system 340. As such, the networked workstation 342 may have a display 344 and a keyboard 346. The networked workstation 342 includes a processor 348 that is commercially available to run a commercially-available operating system. The networked workstation 342 may be able to provide the operator interface that enables scan prescriptions to be entered into the MRI system 300.

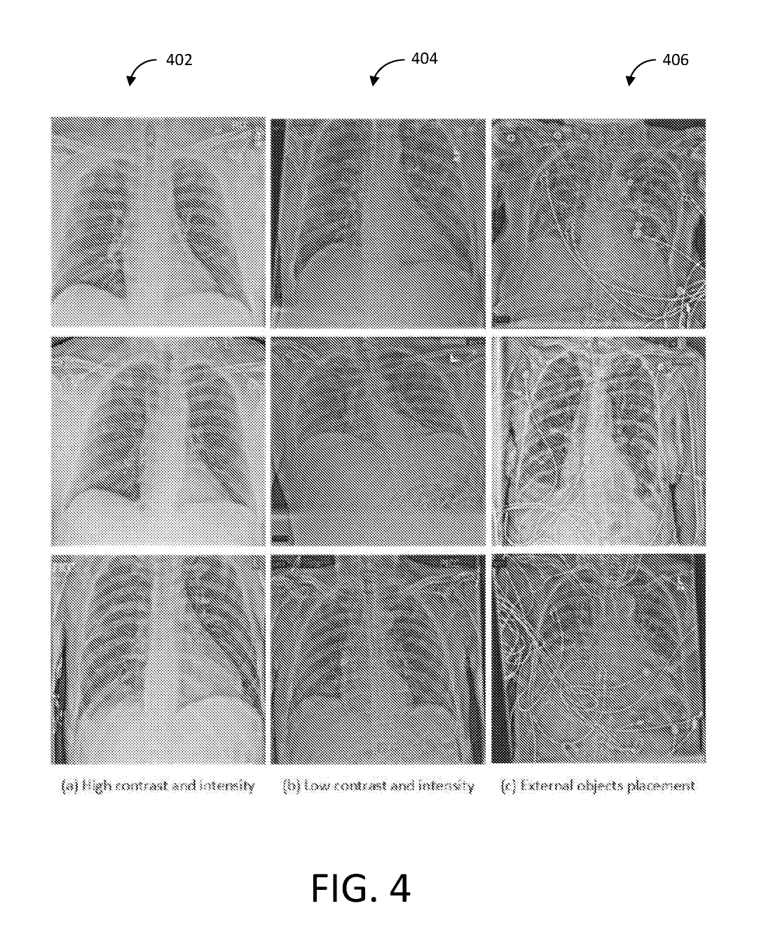

The systems and methods for object (e.g., PICC line) detection that will be described herein may identify the location of PICC lines and other items within CXR images of varying quality and content. FIG. 4 shows examples of chest x-ray radiograph (CXR) images depicting various patients having inserted PICC lines. CXR images may vary in both contrast and intensity. For example, CXR images 402 have a relatively high contrast and intensity, while CXR images 404 have comparatively low contrast and intensity. Additionally, CXR images may vary in terms of placement of external objects. For example, CXR images 406 include external objects including various lines, threads, and tubes, which may impede a radiologist's ability to accurately determine the location of the PICC line.

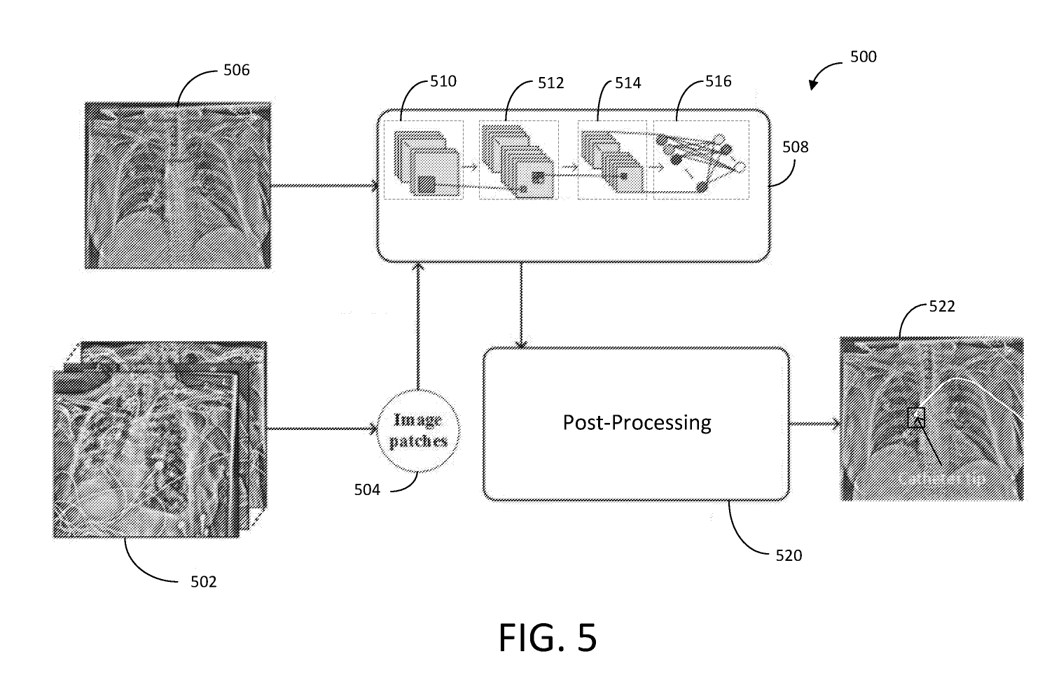

FIG. 5 shows a system architecture 500 for the training and implementation of a neural network 508, which may be used for target object detection in medical images, including PICC line location and catheter tip location detection.

Neural network 508 may be trained using multiple (e.g., 400) CXR images 502 (e.g., CXR images 402, 404, and 406, FIG. 4). CXR images 502 may be varied with respect to contrast and intensity as well as with respect to the number of external objects included in each image. In this way, neural network 508 may be trained to identify the locations of PICC lines, catheter tips, or other specified objects in CXR images under a variety of conditions.

Non-processed CXR images can sometimes be hazy and may have low pixel contrast, which may impede a neural network's ability to discriminate a PICC line from similarly appearing objects. Thus, CXR images 502 may undergo preprocessing in order to standardize the quality, orientation, and dimensions of each of the CXR images 502, which may improve the ability of neural network 508 to learn to identify significant and invariant features of PICC lines. For example, histogram equalization (e.g., Contrast Limited Adaptive Histogram Equalization) may be applied to CXR images 502 to achieve consistency in image contrast. Second, CXR images 502 may then be zero-padded in order to equalize their widths and heights while preserving their respective aspect ratios. Next, CXR images 502 may be resized to a predetermined set of dimensions (e.g., 1024 pixels×1024 pixels). In some embodiments, a bilateral filter may be applied to CXR images 502 for de-noising and edge enhancement.

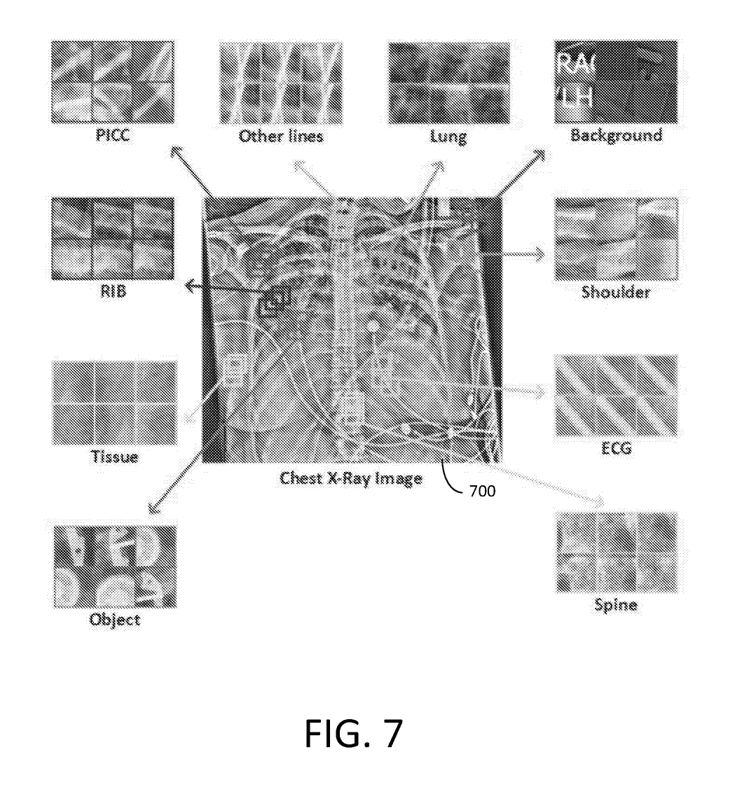

After this pre-processing, image patches 504 may be sampled from each of CXR images 502. Image patches 504 may each be similarly dimensioned (e.g., 96 pixels×96 pixels) and may each be associated with particular objects located within the region of interest of CXR images 502. An example of different image patch classifications is shown in FIG. 7. CXR image 700 may be sampled to extract image patches corresponding to numerous classes, including PICC lines, other lines, lungs, rib bones, shoulder bones, tissue, electro cardiogram (ECG) wires, spinal bones, or other objects.

Returning now to FIG. 5, neural network 508 may receive image patches 504 during training. A stochastic gradient descent optimizer (e.g., having a mini-batch size of 1024, a base learning rate of 0.005, and a momentum term of 0.9) may train all layers of neural network 508.

Neural network 508 may be a deep convolutional neural network that includes an input layer 510, a convolutional layer 512, a pooling layer 514, and a fully connected layer 516. Neural network 508, for example, may be implemented as software instructions stored on a non-transitory computer-readable storage medium and executed by a hardware processor in a computer system. Each unit of data of each layer of neural network 508 may be referred to herein as a neuron, and groups of neurons may be referred to as neuron clusters. When identifying the location of an PICC line and catheter tip in a CXR image 506, the image data (e.g., pixel data) of CXR image 506 may be arranged in a matrix at input layer 510 of neural network 508. Convolutional processing is performed on the matrix of image data using convolutional layer 512. For example, convolutional layer 512 may apply a predetermine number of filters (e.g., convolutional filters) to the matrix of image data (e.g., to the outputs of input layer 510). A pooling operation is then applied to the outputs of convolutional layer 512 using pooling layer 514. For example, pooling layer 514 may combine the output of a neuron cluster (e.g., having predetermined dimensions) of convolutional layer 512 into a single neuron of a pooled matrix. Pooling layer 514 may perform this combining operation on all neuron clusters of the convolved matrix. Fully connected layer 516 is then applied to the output of pooling layer 514 to produce a classification image in which a predicted location of one or more target objects (e.g., a PICC line location and/or catheter tip location) are identified. Here, “fully connected” refers to the fact that each neuron of fully connected layer 516 is respectively connected to a corresponding neuron of pooling layer 514. For example, the trained activation function may include any of hyperbolic tangent, sigmoidal, or rectified linear units activation functions or any other applicable activation functions.

In order to derive useful information from the classification image that is output by neural network 508, post-processing engine 520 may be used to analyze the classification engine. Post-processing engine 520, for example, may be implemented as software instructions stored on a non-transitory computer-readable storage medium and executed by a hardware processor in a computer system.

Post-processing engine 520 may first extract line shapes corresponding to one or more target objects from the classification image. For example, a generalized Hough transform may be applied to the classification image, which may use a voting procedure to extract line shapes corresponding to the target object(s) and to filter out false-positives that may occur (e.g., as a result of similarities between bone edges that may resemble PICC lines).

After the line shape extraction has been performed, a refined mask corresponding to the location of the target object(s) in CXR image 506 is generated. For example, the refined mask may be generated by merging significant contours detected near the extracted line shapes. This mask may be then be superimposed on CXR image 506 to produce an output image 522, so as to highlight the location(s) of the target object(s) in output image 522. Different colors and/or shapes may be used to indicate different target object classes in output image 522. For example, the location of a PICC line in output image 522 may be highlighted in a first color, while a catheter tip for the PICC line may be highlighted in a second color and/or may be indicated as lying within a region outlined by a square of the second color.

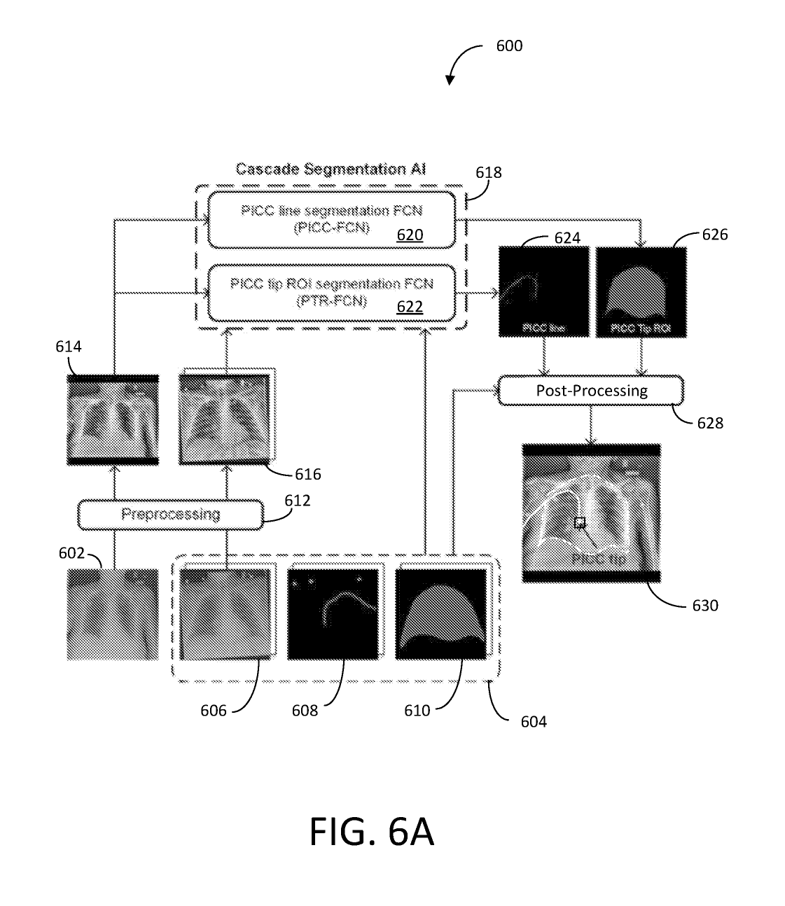

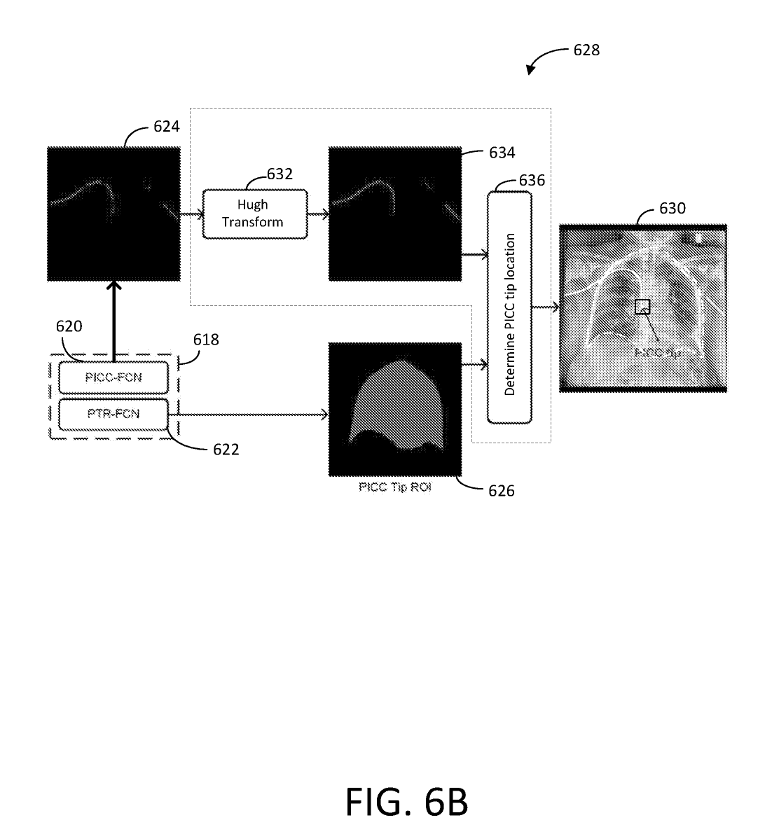

FIG. 6 shows a system architecture 600 for the training and implementation of a cascade segmentation artificial intelligence (AI) 618, which may be used for target object detection in medical images, including PICC line location and catheter tip location and region of interest (ROI) detection.

Cascade segmentation AI 618 may include multiple fully convolutional networks (FCNs), with each FCN being trained to identify the location of a different class of target object. For example, cascade segmentation AI may include a PICC line segmentation FCN 620 (sometimes referred to herein as PICC-FCN) that is trained to detect the location of a PICC line in a CXR image, and may include a PICC tip ROI segmentation FCN 622 (sometimes referred herein to as PTR-FCN 622) that is trained to determine a region of interest in which the tip the PICC line may be located (e.g., within a patient's chest cavity). A FCN is fully convolutional, meaning that it includes only convolutional layers that apply learning filters at each stage of the FCN, and does not include any full-connected layers. By using FCNs in cascade segmentation AI 618, images and ground truth labels may be processed in one forward pass for pixel-wise segmentation from any-sized input images.

The FCNs of cascade segmentation AI 618 may be trained using training and validation dataset 604, which includes training CXR images 606 used to train cascade segmentation AI 618, and ground truth labels 608 and 610 used to verify the outputs of cascade segmentation AI 618. The FCNs of cascade segmentation AI 618 may be trained end-to-end, pixel-to-pixel, with training CXR images 606 and ground truth labels 608 and 610, and may then be deployed whole-image-at-a-time. Once cascade segmentation AI 618 has undergone training, it may be used to predict the locations of one or more target objects in a CXR image 602.

During normal operation pre-processing engine 612 may normalize contrast and dimensions of CXR image 602 to generate a pre-processed CXR image 614. During training, pre-processing engine 512 may normalize contrast and dimensions of training CXR images 606 and to generate pre-processed training CXR images 616. Pre-processing engine 612 may be implemented as software instructions stored on a non-transitory computer-readable storage medium and executed by a hardware processor in a computer system. First, histogram equalization (e.g., Contrast Limited Adaptive Histogram Equalization (CLAHE)) may be applied to a CXR image by pre-processing engine 612. In this way, pre-processing engine 612 may achieve consistency in image contrast for CXR images provided to cascade segmentation AI 618. Next, the CXR image may be zero-padded by pre-processing engine 612. In this way, pre-processing engine 612 may equalize widths and heights for CXR images provided to cascade segmentation AI 618, while preserving the aspect ratios of these images.

PICC-FCN 620 and PICC tip ROI segmentation FCN 622 may each receive pre-processed CXR images produced by pre-processing engine 612 (e.g., pre-processed CXR image 614 during normal operation, or pre-processed training CXR images 616 during training). For a given CXR image, PICC-FCN 620 may generate a PICC line prediction image 624, and PTR-FCN 622 may generate a PICC line tip (e.g., catheter tip) ROI prediction image 626, both of which may subsequently be processed by post-processing engine 628.

Post-processing engine 628 is shown in greater detail in FIG. 6B. Post-processing engine 628 may receive PICC line prediction image 624 and PICC line tip ROI prediction image 626 from cascade segmentation AI 618. Post-processing engine 628 may apply a probabilistic Hough line transform algorithm to PICC line prediction image 624 at block 632 in order to remove any predicted PICC line locations shown in image 624 that were detected erroneously. Additionally, the Hough line transform algorithm may effectively merge significant contours near the predicted PICC line locations in order to generate a filtered PICC line prediction image 634 having a comparatively smoothly curved PIC line trajectory. At block 636, the location of the tip of the PICC line may be predicted by post-processing engine 628 based on the PICC line tip ROI prediction image 626 and on the filtered PICC line tip prediction image 634, and post-processing engine 628 may generate a mask and then superimpose mask on the pre-processed CXR image (e.g., CXR image 614 or one of training CXR images 616) to produce an output image 630. The mask may highlight the predicted locations of the PICC line, the PICC line tip ROI, and the PICC line tip itself in respectively different colors.

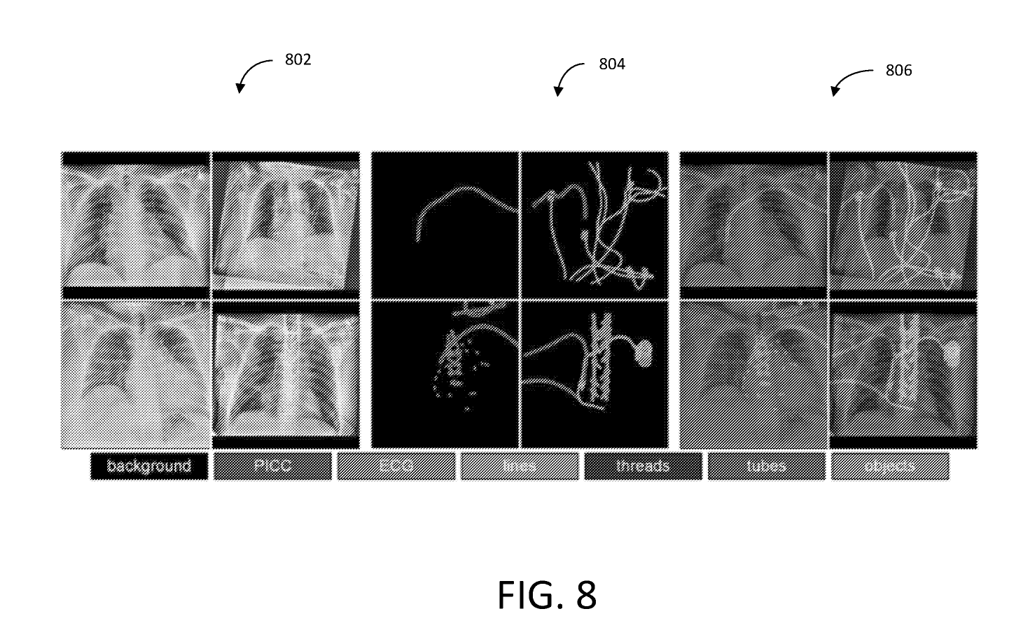

It should be noted that the system architectures for target object detection described above in connection with FIGS. 5, 6A, and 6B are not limited to the detection of PICC lines. FIG. 8 shows examples of original images 802, ground truth labels 804, and output images 806, illustrating various classes of target objects that may be identified with these system architectures. As shown, the locations of PICC lines, electrocardiogram (ECG) lines, threads, tubes, and other objects (e.g., medical implants) may be segmented and identified using embodiments of the present disclosure.

What is claimed is:

1. A medical imaging system comprising:
an image sensor configured to acquire image data from a patient to produce a captured image;
a processor configured to receive the image data from the image sensor, to determine a location of a peripherally inserted central catheter (PICC) line in the image, and to generate an output image in which the location of the PICC line is highlighted; and
a display configured to display the output image.

2. The medical imaging system of claim 1 wherein the image sensor includes at least one of:
a radio frequency (RF) system of a magnetic resonance imaging (MRI) system;
an x-ray detector of a computed tomography (CT) system; and
a gamma ray detector of an emission tomography system.

3. The medical imaging system of claim 1, wherein the processor is configured to determine the location of the PICC line using a first trained neural network.

4. The medical imaging system of claim 3, wherein the processor is further configured to determine a region of interest for a location of a tip of the PICC line using a second trained neural network.

5. The medical imaging system of claim 4, wherein the processor is further configured to determine the location of the tip of the PICC line based on the location of the PICC line and the region of interest, and to generate a mask that includes the location of the tip of the PICC line, the location of the region of interest, and the location of the PICC line, wherein the output image comprises the mask superimposed over the captured image.

6. A system comprising:
an input configured to receive image data from an imaging system configured to generate the image data, wherein the image data corresponds to a captured image; and
a processor configured to receive the image data from the input, to determine a location of a peripherally inserted central catheter (PICC) line in the captured image, and to generate an output image in which the location of the PICC line is highlighted.

7. The system of claim 6, wherein the processor is configured to determine the location of the PICC line by processing the image data with a first neural network to produce a PICC line prediction image.

8. The system of claim 7, wherein the first neural network comprises a fully convolutional neural network that includes a plurality of convolutional layers.

9. The system of claim 8, wherein the processor is further configured to determine the location of a region of interest (ROI) for a location of a tip of the PICC line by processing the image data with a second neural network to produce a ROI prediction image, wherein the first and second neural networks are included in a cascade segmentation artificial intelligence.

10. The system of claim 9, wherein the processor is further configured to apply a Hough transform to the PICC line prediction image to produce a filtered PICC line prediction image.

11. The system of claim 10, wherein the processor is further configured to determine the location of the tip of the PICC line based on the filtered PICC line prediction image and the ROI prediction image.

12. The system of claim 11, wherein the processor is further configured to produce an output image by:
generating a mask based on the filtered PICC line prediction image, the ROI prediction image, and the determined location of the tip of the PICC line; and
superimposing the mask over the captured image to produce the output image.

13. The system of claim 6, wherein the imaging system includes at least one of:
a radio frequency (RF) system of a magnetic resonance imaging (MRI) system;
an x-ray detector of a computed tomography (CT) system; and
a gamma ray detector of an emission tomography system.

14. A method comprising:
generating, with an imaging system, image data that corresponds to a captured image;
receiving, with a processor, the image data from the imaging system; and
executing, with the processor, instructions for determining a location of a peripherally inserted central catheter (PICC) line in the captured image, and generating an output image in which the location of the PICC line is highlighted.

15. The method of claim 14, wherein determining the location of the PICC line in the captured image comprises:
determining the location of the PICC line in the captured image by processing the image data with a first neural network to produce a PICC line prediction image.

16. The method of claim 15, further comprising:
executing, with the processor, instructions for determining a location of a region of interest (ROI) for a location of a tip of the PICC line by processing the image data with a second neural network to produce a ROI prediction image, wherein the first and second neural networks are included in a cascade segmentation artificial intelligence.

17. The method of claim 16, wherein the first neural network and the second neural network comprise fully convolutional neural networks that each include a plurality of convolutional layers.

18. The method of claim 17, further comprising:
executing, with the processor, instructions for applying a Hough transform to the PICC line prediction image to produce a filtered PICC line prediction image.

19. The method of claim 18, further comprising:
executing, with the processor, instructions for determining the location of the tip of the PICC line based on the filtered PICC line prediction image and the ROI prediction image.

20. The method of claim 19, further comprising:
executing, with the processor, instructions for generating a mask based on the filtered PICC line prediction image, the ROI prediction image, and the determined location of the tip of the PICC line; and
executing, with the processor, instructions for superimposing the mask over the captured image to produce the output image.
